(19) United States
(12) Patent Application Publication
(10) Pub. No.: US 2016/0157835 A1
(43) Pub. Date: Jun. 9, 2016

(54) APPARATUS AND METHOD FOR A URINE SAMPLE COLLECTION DEVICE

(71) Applicants: Jacqueline Fountain, Ooltewah, TN (US); Raymond Hunter, Ooltewah, TN (US)

(72) Inventors: Jacqueline Fountain, Ooltewah, TN (US); Raymond Hunter, Ooltewah, TN (US)

(21) Appl. No.: 14/959,655

(22) Filed: Dec. 4, 2015

Related U.S. Application Data
(60) Provisional application No. 62/124,116, filed on Dec. 8, 2014.

(51) Int. Cl. A61B 10/00

(52) U.S. Cl. CPC A61B 10/007; A61B 2560/0406; A61B 2560/0443

(57) ABSTRACT

A urine sample collection device adapted to be removably connected to a urine sample container and comprising a funneling portion having a side wall defining an upper opening and a lower opening, a cylindrical portion disposed adjacent to the lower opening of the funneling portion about a vertical axis, and a handle disposed on the side wall of the funneling portion and extending radially away from the vertical axis. Preferably, the upper opening of the funneling portion is larger than the lower opening, and the cylindrical portion is adapted to be removably attached to the urine sample container. A method for collecting a urine sample comprising providing a urine sample collection device and removably attaching the urine sample collection device to the urine sample container.

### CROSS-REFERENCES TO RELATED APPLICATIONS/PATENTS

This application relates back to and claims the benefit of priority from U.S. Provisional Application for Patent Ser. No. 62/124,116 entitled “Dry Hands Urine Specimen Device and Dry Hands Urine Specimen Lid” and filed on Dec. 8, 2014.

### FIELD OF THE INVENTION

The present invention relates generally to medical devices, and particularly to urine sample collection devices.

### BACKGROUND AND DESCRIPTION OF THE PRIOR ART

It is known to use a plastic container for the collection of human urine to be used for medical testing and analysis. Conventional containers, however, suffer from one or more disadvantages. For example, conventional containers are difficult to accurately hold in place to collect urine. Conventional containers also have undesirably small openings for receiving the urine sample. Conventional collection containers also do not direct or funnel urine toward the opening of the container. As a result, the collection process is messy and often involves urine spilling outside the container and onto the user. Conventional containers also require the user's hands to be undesirably close to the urine sample, and therefore, they increase the likelihood of contamination of the urine sample through contact with the user's hands.

It would be desirable, therefore, if an apparatus and method for a urine sample collection device could be provided that would be easier for the user to accurately hold in place to collect urine. It would also be desirable if such an apparatus and method for a urine sample collection device could be provided that would provide a sufficiently large opening for receiving the urine sample. It would be further desirable if such an apparatus and method for a urine sample collection device could be provided that would direct the urine flow toward a funneled area to deposit the urine inside the collection container. It would be still further desirable if such apparatus and method for a urine sample collection device could be provided that would minimize or eliminate spillage of urine outside the collection container. In addition, it would be desirable if such an apparatus and method for a urine sample collection device could be provided that would allow the user's hands to be spaced a distance apart from the urine sample, thereby minimizing or eliminating the risk of contaminating the urine sample through contact with the user's hands.

Additional advantages of the preferred embodiments of the invention will become apparent from an examination of the drawings and the ensuing description.

### ADVANTAGES OF THE PREFERRED EMBODIMENT OF THE INVENTION

Accordingly, it is an advantage of the preferred embodiments of the invention described and claimed herein to provide an apparatus and method for a urine sample collection device that allows the user to accurately hold the device in place to collect a urine sample. It is also an advantage of the preferred embodiments of the invention described and claimed herein to provide an apparatus and method for a urine sample collection device that provides a sufficiently large opening for receiving the urine sample. It is another advantage of the preferred embodiments of the invention described and claimed herein to provide an apparatus and method for a urine sample collection device that directs the urine flow toward a funneled area to deposit the urine inside the collection container. It is yet another advantage of the preferred embodiments of the invention described and claimed herein to provide an apparatus and method for a urine sample collection device that minimizes or eliminates spillage of urine outside the collection container. It is still another advantage of the preferred embodiments of the invention described and claimed herein to provide an apparatus and method for a urine sample collection device that allows the user's hands to be spaced a distance apart from the urine sample, thereby minimizing or eliminating the risk of contamination of the urine sample through contact between the urine sample and the user's hands.

### SUMMARY OF THE INVENTION

The apparatus of the invention comprises a urine sample collection device adapted to be removably connected to a urine sample container. The preferred urine sample collection device comprises a funneling portion having a side wall defining an upper opening and a lower opening, a cylindrical portion which is disposed adjacent to the lower opening of the funneling portion about a vertical axis, and a handle which is disposed on the side wall of the funneling portion and extends radially away from the vertical axis. In the preferred embodiments of the apparatus, the upper opening of the funneling portion is larger than the lower opening, and the cylindrical portion is adapted to be removably attached to the urine sample container.

The method of the invention comprises a method for collecting a urine sample. The preferred method comprises providing a urine sample collection device adapted for use with a urine sample container. The preferred urine sample collection device comprises a funneling portion having a side wall defining an upper opening and a lower opening, a cylindrical portion which is disposed adjacent to the lower opening of the funneling portion about a vertical axis, and a handle which is disposed on the side wall of the funneling portion and extends radially away from the vertical axis. In the preferred embodiments of the apparatus, the upper opening of the funneling portion is larger than the lower opening, and the cylindrical portion is adapted to be removably attached to the urine sample container. The preferred method also comprises removably attaching the urine sample collection device to the urine sample container.

### BRIEF DESCRIPTION OF THE DRAWINGS

The presently preferred embodiments of the invention are illustrated in the accompanying drawings, in which like reference numerals represent like parts throughout, and in which:

FIG. 1 is a perspective view of the preferred embodiment of the urine sample collection device in accordance with the present invention shown attached to an exemplary urine sample collection container.

FIG. 2 is a perspective view of the preferred urine sample collection device illustrated in FIG. 1 shown detached from an exemplary urine sample collection container.

FIG. 3 is a top view of the preferred urine sample collection device illustrated in FIGS. 1-2.

FIG. 4 is a right side view of the preferred urine sample collection device illustrated in FIGS. 1-3.

FIG. 5 is a front view of the preferred urine sample collection device illustrated in FIGS. 1-4.

FIG. 6 is a top perspective view of a first alternative embodiment of the urine sample collection device in accordance with the present invention shown with an exemplary urine sample collection container.

FIG. 7 is a bottom perspective view of the first alternative embodiment of the urine sample collection device illustrated in FIG. 6.

FIG. 8 is a top view of the first alternative embodiment of the urine sample collection device illustrated in FIGS. 6-7.

FIG. 9 is a right side view of the first alternative embodiment of the urine sample collection device illustrated in FIGS. 6-8.

FIG. 10 is a back view of the first alternative embodiment of the urine sample collection device illustrated in FIGS. 6-9.

FIG. 11 is an enlarged partial sectional right side view of the cylindrical portion of the first alternative embodiment of the urine sample collection device illustrated in FIGS. 6-10.

FIG. 12 is an enlarged partial sectional perspective view of the cylindrical portion of the first alternative embodiment of the urine sample collection device illustrated in FIGS. 6-11.

### DESCRIPTION OF THE PREFERRED EMBODIMENTS OF THE INVENTION

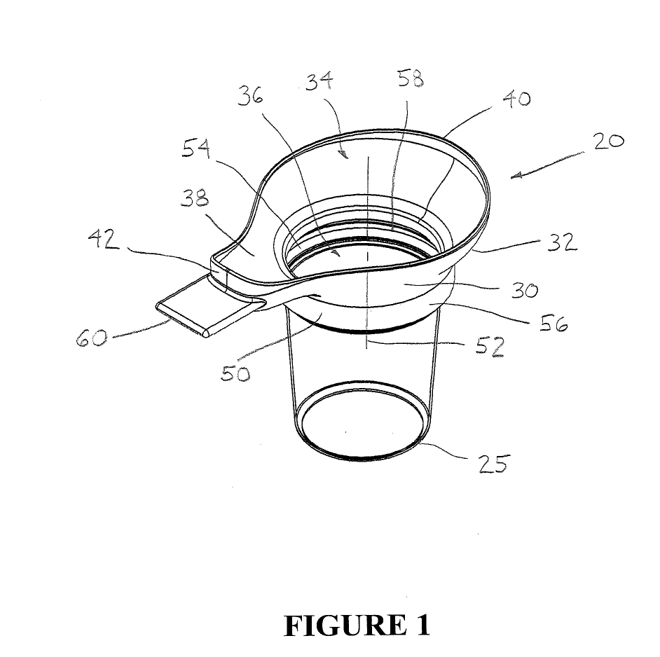

Referring now to the drawings, the preferred embodiments of the urine sample collection device in accordance with the present invention are illustrated by FIGS. 1 through 12. As shown in FIGS. 1-12, the preferred embodiments of an apparatus and method for a urine sample collection device are adapted to allow a user to accurately hold the device in place to collect a urine sample. The preferred embodiments also provide a sufficiently large opening for receiving the urine sample. The preferred embodiments also direct the urine flow toward a funneled area to deposit the urine inside the collection container. The preferred embodiments further minimize or eliminate spillage of urine outside the collection container. The preferred embodiments still further allow the user's hands to be spaced a distance apart from the urine sample, thereby minimizing or eliminating the risk of contamination of the urine sample through contact between the urine sample and the user's hands.

Referring now to FIG. 1, a perspective view of the preferred embodiment of the urine sample collection device in accordance with the present invention is illustrated. More particularly, the preferred urine sample collection device is shown attached to an exemplary urine sample collection container. As shown in FIG. 1, the preferred urine sample collection device is designated generally by reference numeral 20 and exemplary urine sample collection container is designated generally by reference numeral 25. Preferred urine sample collection device 20 is adapted to be removably connected to container 25 and comprises funneling portion 30 having side wall 32 which defines upper opening 34 and lower opening 36. Preferred upper opening 34 is generally pear-shaped and preferred lower opening 36 is generally circular. In the preferred urine sample collection device 20, upper opening 34 of funneling portion 30 is larger than lower opening 36. Preferably, side wall 32 of funneling portion 30 also defines elongated portion 38, first raised portion 40 and second raised portion 42. While FIG. 1 illustrates the preferred configuration and arrangement of the funneling portion of the urine sample collection device, it is contemplated within the scope of the invention that the funneling portion of the urine sample collection device may be of any suitable configuration and arrangement.

Still referring to FIG. 1, preferred urine sample collection device 20 also comprises cylindrical portion 50 which is disposed adjacent to lower opening 36 of funneling portion 30 about vertical axis 52. Preferred elongated portion 38 extends radially away from vertical axis 52. Preferred cylindrical portion 50 comprises inner surface 54 and outer surface 56 which is spaced apart from the inner surface. Preferred cylindrical portion 50 also comprises threads 58 disposed on inner surface 54. In the preferred urine sample collection device 20, cylindrical portion 50 is adapted to be removably attached to urine sample container 25. While FIG. 1 illustrates the preferred configuration and arrangement of the cylindrical portion of the urine sample collection device, it is contemplated within the scope of the invention that the cylindrical portion may be of any suitable configuration and arrangement.

Still referring to FIG. 1, preferred urine sample collection device 20 further comprises handle 60 which is disposed on side wall 32 of funneling portion 30 and extends radially away from vertical axis 52. Preferably, handle 60 is disposed on elongated portion 38 of side wall 32 of funneling portion 30 in an area adjacent to second raised portion 42. More preferably, handle 60 is disposed on second raised portion 42 of elongated portion 38 of side wall 32 of funneling portion 30. Preferably first raised portion 40 of side wall 32 of funneling portion 30 is in an area spaced apart from handle 60. While FIG. 1 illustrates the preferred configuration and arrangement of the handle of the urine sample collection device, it is contemplated within the scope of the invention that the handle may be of any suitable configuration and arrangement.

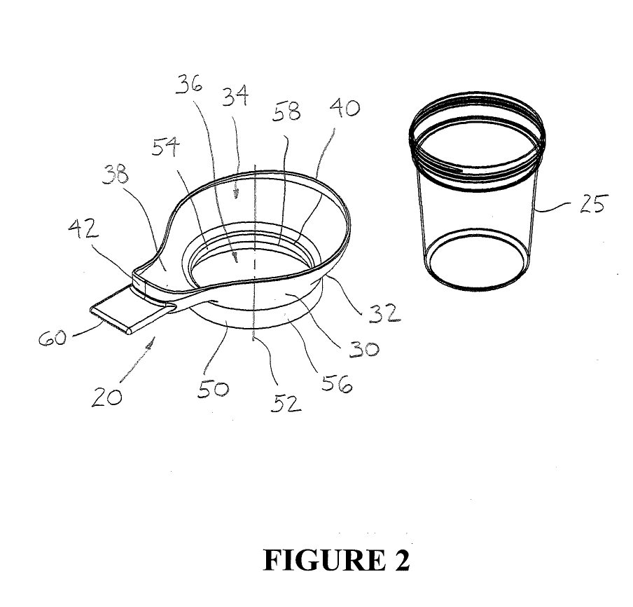

Referring now to FIG. 2, a perspective view of preferred urine sample collection device 20 is illustrated. More particularly, preferred urine sample collection device 20 is shown detached from exemplary urine sample collection container 25. As shown in FIG. 2, preferred urine sample collection device 20 comprises funneling portion 30 having side wall 32 which defines upper opening 34, lower opening 36, elongated portion 38, first raised portion 40, and second raised portion 42. Preferred urine sample collection device 20 also comprises cylindrical portion 50 having inner surface 54, outer surface 56, and threads 58. Preferred urine sample collection device 20 further comprises handle 60.

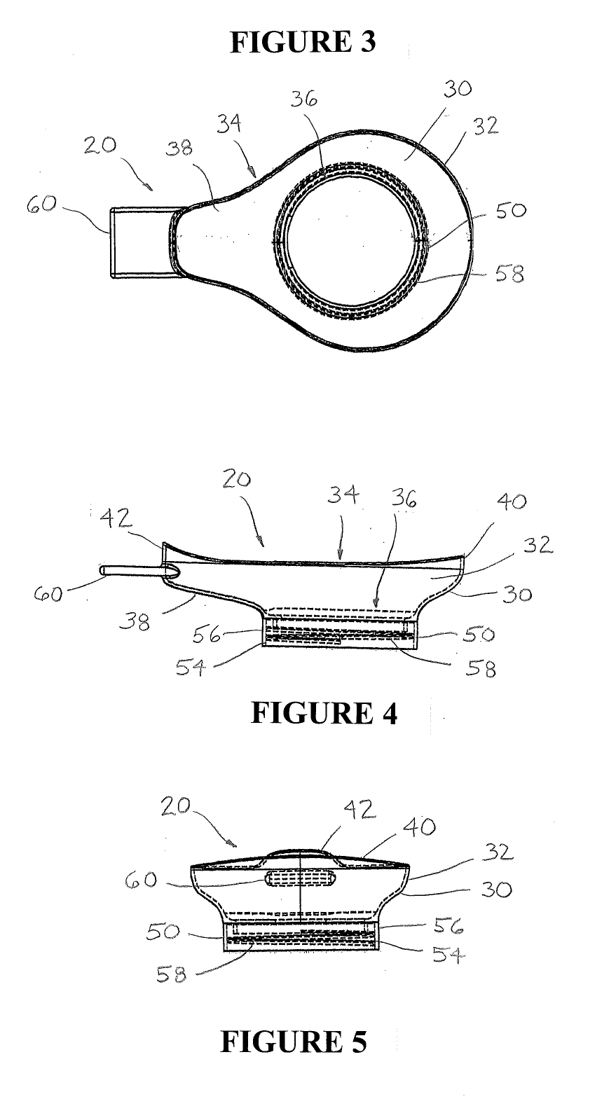

Referring now to FIG. 3, a top view of preferred urine sample collection device 20 is illustrated. As shown in FIG. 3, preferred urine sample collection device 20 comprises funneling portion 30 having side wall 32 defiling pear-shaped upper opening 34, circular lower opening 36, and elongated portion 38. Preferred urine sample collection device 20 also comprises cylindrical portion 50 having threads 58 and handle 60.

Referring now to FIG. 4, a right side view of preferred urine sample collection device 20 is illustrated. As shown in FIG. 4, preferred urine sample collection device 20 comprises funneling portion 30 having side wall 32 which defines upper opening 34, lower opening 36, elongated portion 38, first raised portion 40, and second raised portion 42. Preferred urine sample collection device 20 also comprises cylindrical portion 50 having inner surface 54, outer surface 56, and threads 58. Preferred urine sample collection device 20 further comprises handle 60.

Referring now to FIG. 5, a front view of preferred urine sample collection device 20 is illustrated. As shown in FIG. 5, preferred urine sample collection device 20 comprises funneling portion 30 having side wall 32 which defines first raised portion 40 and second raised portion 42. Preferred urine sample collection device 20 also comprises cylindrical portion 50 having inner surface 54, outer surface 56, and threads 58. Preferred urine sample collection device 20 further comprises handle 60.

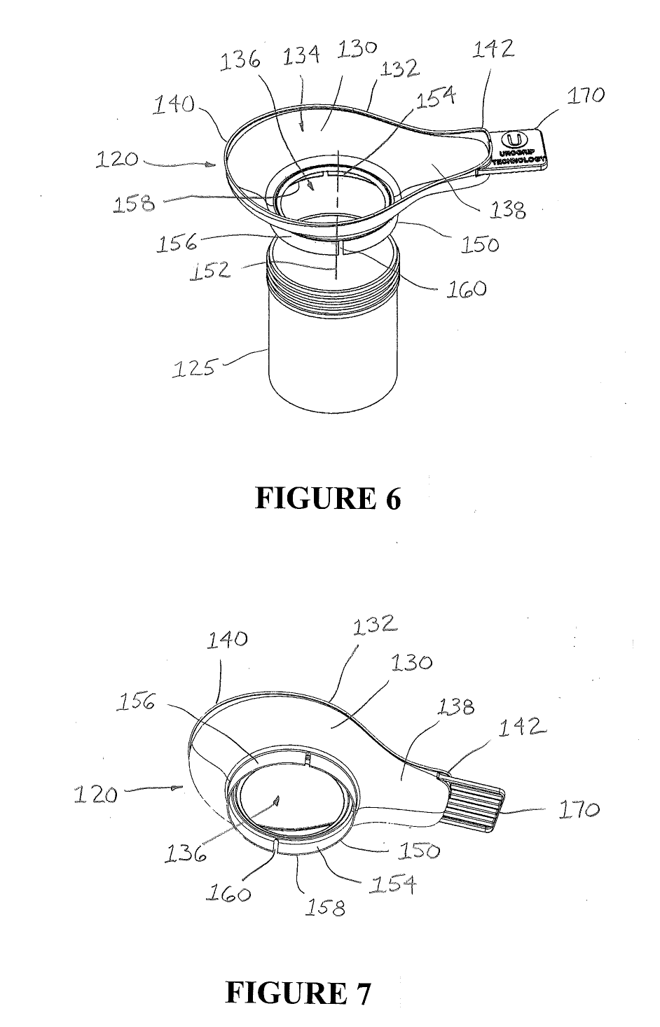

Referring now to FIG. 6, a top perspective view of a first alternative embodiment of the urine sample collection device in accordance with the present invention is illustrated. More particularly, the first alternative embodiment of the urine sample collection device is shown with an exemplary urine sample collection container. As shown in FIG. 6, the preferred urine sample collection device is designated generally by reference numeral 120 and exemplary urine sample collection container is designated generally by reference numeral 125. Preferred urine sample collection device 120 is adapted to be removably connected to container 125 and comprises funneling portion 130 having side wall 132 which defines upper opening 134 and lower opening 136. Preferred upper opening 134 is generally pear-shaped and preferred lower opening 136 is generally circular. In the preferred urine sample collection device 120, upper opening 134 of funneling portion 130 is larger than lower opening 136. Preferably, side wall 132 of funneling portion 130 also defines elongated portion 138, first raised portion 140, and second raised portion 142. While FIG. 6 illustrates the preferred configuration and arrangement of the funneling portion of the urine sample collection device, it is contemplated within the scope of the invention that the funneling portion of the urine sample collection device may be of any suitable configuration and arrangement.

Still referring to FIG. 6, preferred urine sample collection device 120 also comprises cylindrical portion 150 which is disposed adjacent to lower opening 136 of funneling portion 130 about vertical axis 152. Preferred elongated portion 138 extends radially away from vertical axis 152. Preferred cylindrical portion 150 comprises inner surface 154 and outer surface 156 which is spaced apart from the inner surface. Preferred cylindrical portion 150 also comprises lip 158 which is disposed on inner surface 154 and adapted to engage the threads of container 125. Preferred cylindrical portion 150 further comprises slots 160 which are adapted to permit urine sample collection device 120 to be used with different-sized urine sample containers. In the preferred urine sample collection device 120, cylindrical portion 150 is adapted to be removably attached to urine sample container 125. While FIG. 6 illustrates the preferred configuration and arrangement of the cylindrical portion of the urine sample collection device, it is contemplated within the scope of the invention that the cylindrical portion may be of any suitable configuration and arrangement. It is also contemplated within the scope of the invention that fewer or more than two slots may be provided in the cylindrical portion of the urine sample collection device.

Still referring to FIG. 6, preferred urine sample collection device 120 further comprises handle 170 which is disposed on side wall 132 of funneling portion 130 and extends radially away from vertical axis 152. Preferably, handle 170 is disposed on elongated portion 138 of side wall 132 of funneling portion 130 in an area adjacent to second raised portion 142. More preferably, handle 170 is disposed on second raised portion 142 of elongated portion 138 of side wall 132 of funneling portion 130. Preferably first raised portion 140 of side wall 132 of funneling portion 130 is in an area spaced apart from handle 170. While FIG. 6 illustrates the preferred configuration and arrangement of the handle of the urine sample collection device, it is contemplated within the scope of the invention that the handle may be of any suitable configuration and arrangement.

Referring now to FIG. 7, a bottom perspective view of preferred urine sample collection device 120 is illustrated. As shown in FIG. 7, preferred urine sample collection device 120 comprises funneling portion 130 having side wall 132 which defines lower opening 136, elongated portion 138, first raised portion 140, and second raised portion 142. Preferred urine sample collection device 120 also comprises cylindrical portion 150 having inner surface 154, outer surface 156, lip 158, and slots 160. Preferred urine sample collection device 120 further comprises handle 170.

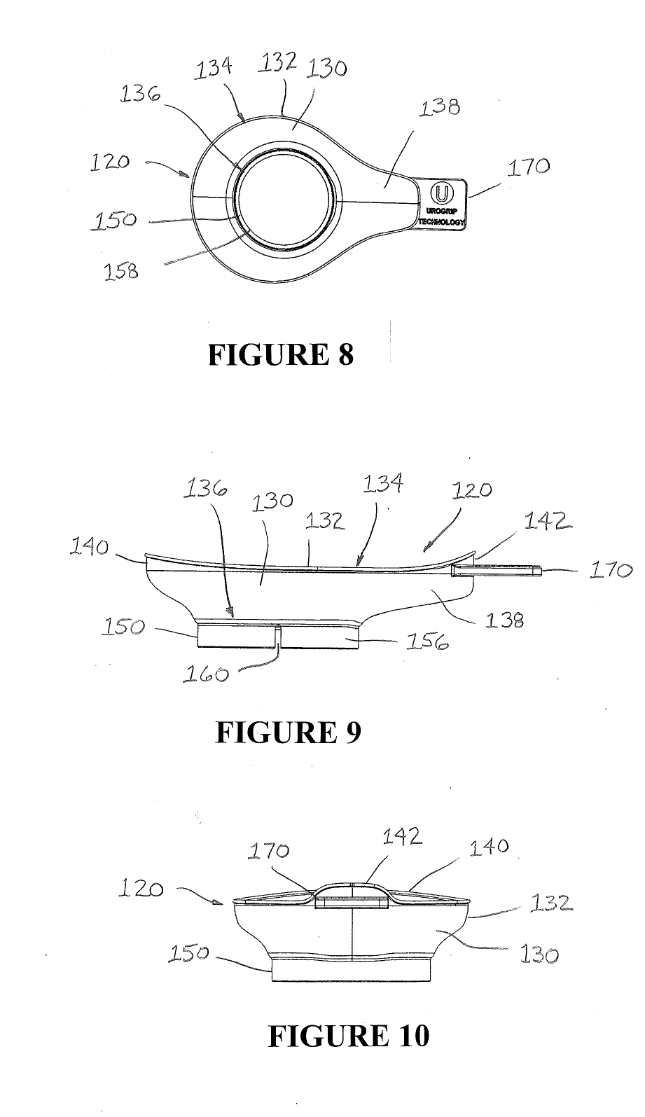

Referring to FIG. 8, a top view of preferred urine sample collection device 120 is illustrated. As shown in FIG. 8, preferred urine sample collection device 120 comprises funneling portion 130 having side wall 132 defining pear-shaped upper opening 134, circular lower opening 136, and elongated portion 138. Preferred urine sample collection device 120 also comprises cylindrical portion 150, lip 158, and handle 170.

Referring now to FIG. 9, a right side view of preferred urine sample collection device 120 is illustrated. As shown in FIG. 9, preferred urine sample collection device 120 comprises funneling portion 130 having side wall 132 which defines upper opening 134, lower opening 136, elongated portion 138, first raised portion 140, and second raised portion 142. Preferred urine sample collection device 120 also comprises cylindrical portion 150 having outer surface 156 and slot 160. Preferred urine sample collection device 120 further comprises handle 170.

Referring now to FIG. 10, a back view of preferred urine sample collection device 120 is illustrated. As shown in FIG. 10, preferred urine sample collection device 120 comprises funneling portion 130 having side wall 132 which defines first raised portion 140 and second raised portion 142. Preferred urine sample collection device 120 also comprises cylindrical portion 150 having outer surface 156. Preferred urine sample collection device 120 further comprises handle 170.

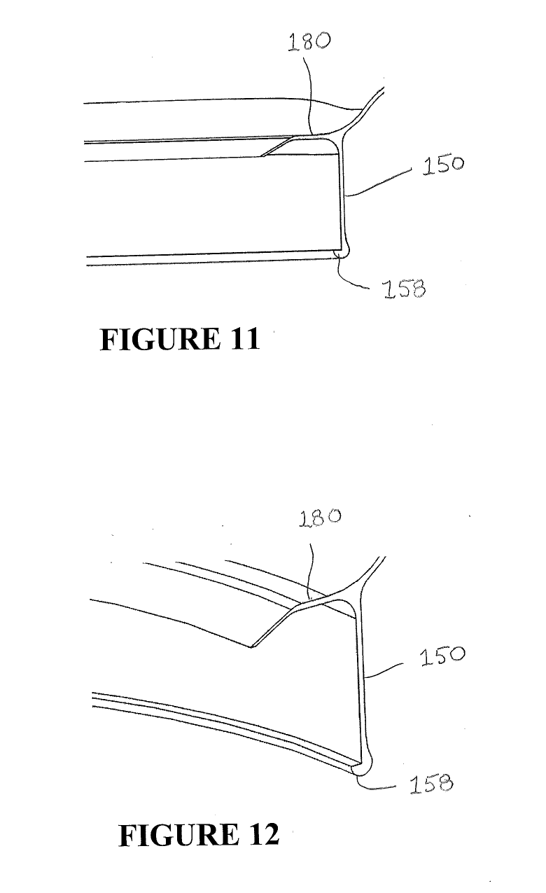

Referring now to FIG. 11, an enlarged partial sectional right side view of cylindrical portion 150 of urine sample collection device 120 is illustrated. As shown in FIG. 11, preferred cylindrical portion 150 comprises lip 158 which extends inwardly toward vertical axis 152 so as to be adapted to engage with the threads of exemplary container 125. Preferably, lip 158 is disposed on the lower portion of cylindrical portion 150. In addition, preferred cylindrical portion comprises rim 180. Preferred rim 180 extends inwardly toward vertical axis 152 so as to be adapted to bear against the top of exemplary container 125 when urine sample collection device 120 is removably attached to the container. Preferably, rim 180 is disposed on the upper portion of cylindrical portion 150.

FIG. 12 is an enlarged partial sectional perspective view of the cylindrical portion of the first alternative embodiment of the urine sample collection device illustrated in FIGS. 6-11. As shown in FIG. 12, preferred cylindrical portion 150 comprises lip 158 and rim 180.

The invention also comprises a method for collecting a urine sample. The preferred method comprises providing a urine sample collection device adapted for use with a urine sample container. The preferred urine sample collection device comprises a funneling portion having a side wall defining an upper opening and a lower opening, a cylindrical portion which is disposed adjacent to the lower opening of the funneling portion about a vertical axis, and a handle which is disposed on the side wall of the funneling portion and extends radially away from the vertical axis. In the preferred urine sample collection device, the upper opening of the funneling portion is larger than the lower opening, and the cylindrical portion is adapted to be removably attached to the urine sample container. The preferred method also comprises removably attaching the urine sample collection device to the urine sample container and removing the urine sample collection device from the urine sample container.

In operation, several advantages of the preferred embodiments of the apparatus and method for a urine sample collection device are achieved. For example, the preferred embodiments of the urine sample collection device are easily removed from and replaced onto different-sized urine sample collection containers. In addition, the preferred embodiments of the urine sample collection device allow a user to more easily place the device in an accurate position and collect a urine sample without spillage or contamination of the sample.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations, and the same are intended to be comprehended within the meaning and range of equivalents of the appended claims.

What is claimed:

1. A urine sample collection device adapted to be removably connected to a urine sample container, said urine sample collection device comprising:
(a) a funneling portion, said funneling portion having a side wall defining an upper opening and a lower opening;
(b) a cylindrical portion, said cylindrical portion being disposed adjacent to the lower opening of the funneling portion about a vertical axis;
(c) a handle, said handle being disposed on the side wall of the funneling portion and extending radially away from the vertical axis;
wherein the upper opening of the funneling portion is larger than the lower opening; and
wherein the cylindrical portion is adapted to be removably attached to the urine sample container.

2. The urine sample collection device of claim 1 wherein the side wall of the funneling portion defines an elongated portion.

3. The urine sample collection device of claim 2 wherein the elongated portion of the side wall of the funneling portion extends radially away from the vertical axis.

4. The urine sample collection device of claim 2 wherein the handle is disposed on the elongated portion of the side wall of the funneling portion.

5. The urine sample collection device of claim 1 wherein the side wall of the funneling portion defines a first raised portion in an area spaced apart from the handle.

6. The urine sample collection device of claim 1 wherein the side wall of the funneling portion defines a second raised portion in an area adjacent to the handle.

7. The urine sample collection device of claim 1 wherein the upper opening of the funneling portion is generally pear-shaped.

8. The urine sample collection device of claim 1 wherein the lower opening of the funneling portion is generally circular.

9. The urine sample collection device of claim 1 wherein the cylindrical portion comprises in inner surface and an outer surface spaced apart from the inner surface.

10. The urine sample collection device of claim 9 wherein the inner surface of the cylindrical portion comprises threads.

11. The urine sample collection device of claim 1 wherein the cylindrical portion includes a slot.

12. The urine sample collection device of claim 1 wherein the handle is disposed on the second raised portion of the side wall of the funneling portion.

13. A urine sample collection device adapted for use with a urine sample container, said urine sample collection device comprising:
(a) a funneling portion, said funneling portion having a side wall defining an upper opening, a lower opening, a first raised portion, a second raised portion, and an elongated portion;
(b) a cylindrical portion, said cylindrical portion being disposed adjacent to the lower opening of the funneling portion about a vertical axis;
(c) a handle, said handle being disposed on the elongated portion of the side wall of the funneling portion and extending radially from the vertical axis;
wherein the upper opening of the funneling portion is larger than the lower opening; and
wherein the cylindrical portion is adapted to be removably attached to the urine sample container.

14. The urine sample collection device of claim 13 wherein the elongated portion of the side wall of the funneling portion extends radially away from the vertical axis.

15. The urine sample collection device of claim 13 wherein the upper opening of the funneling portion is generally pear-shaped.

16. The urine sample collection device of claim 13 wherein the lower opening of the funneling portion is generally circular.

17. The urine sample collection device of claim 13 wherein the cylindrical portion comprises in inner surface and an outer surface spaced apart from the inner surface.

18. The urine sample collection device of claim 17 wherein the inner surface of the cylindrical portion comprises threads.

19. A method for collecting a urine sample, said method comprising:
(a) providing a urine sample collection device adapted for use with a urine sample container, said urine sample collection device comprising:
(1) a funneling portion, said funneling portion having a side wall defining an upper opening and a lower opening;
(2) a cylindrical portion, said cylindrical portion being disposed adjacent to the lower opening of the funneling portion about a vertical axis;
(3) a handle, said handle being disposed on the side wall of the funneling portion and extending radially away from the vertical axis;
wherein the upper opening of the funneling portion is larger than the lower opening; and wherein the cylindrical portion is adapted to be removably attached to the urine sample container; and
(b) removably attaching the urine sample collection device to the urine sample container.

20. The method of claim 19 further comprising:
(c) removing the urine sample collection device from the urine sample container.
